(19) United States
(12) Patent Application Publication
(10) Pub. No.: US 2012/0197228 A1
(43) Pub. Date: Aug. 2, 2012

(54) URINE ABSORBING ARTICLE

(76) Inventors: Hirofumi Miyake, Mima-gun (JP); Akiko Tatsukawa, Mima-gun (JP)

(21) Appl. No.: 13/501,481

(22) PCT Filed: Nov. 22, 2010
(86) PCT No.: PCT/JP2010/006824

§ 371 Date: Apr. 12, 2012

(30) Foreign Application Priority Data
Nov. 27, 2009 (JP) 2009-270167

(51) Int. Cl. A61F 13/45

(52) U.S. Cl. 604/385.03

(57) ABSTRACT

A urine receiving pad 1 has a substantially rectangular bag body 10. The bag body 10 has a side edge portion 16 extending in a lengthwise direction thereof, and a top edge portion 15 connected to the side edge portion 16 and extending in a widthwise direction perpendicular to the lengthwise direction. In the side edge portion 16, an opening 10P opening in the widthwise direction is provided at the side edge portion 16a which is continuous with the top edge portion 15. Since a male genitalia P is inserted through the opening 10P along the widthwise direction, a bottom portion 10c tends to be positioned below the male genitalia P. This can prevent a portion near the bottom portion 10c from being largely folded even when a diaper, underwear, or the like, is worn. Thus, an overflow of urine from the opening 10P can be effectively suppressed.

### TECHNICAL FIELD

The present invention relates to a urine absorbing article which is attached to a male genitalia to absorb urine excreted from the male genitalia.

### BACKGROUND ART

Conventionally, there has been known a urine absorbing article which is attached to a male genitalia within an outer equipment such as a diaper and underwear and absorbs urine excreted from the male genitalia. As an example of such a urine receiving pad, Japanese Patent Application Laid-Open No. 2004-159724 discloses a urine absorbing article having a pouched shape with an opening through which a male genitalia is inserted into the urine absorbing article.

### SUMMARY OF INVENTION

### Technical Problem

FIG. 10 is a diagram showing a state where a conventional urine receiving pad 100 is attached to a male genitalia. As shown in FIG. 10, in the conventional bag-shaped urine receiving pad 100, an opening 101 through which the male genitalia is inserted is provided at a top edge portion 102 (at the side opposite to a bottom portion 103) of the urine receiving pad 100. Generally, a male genitalia P extends obliquely relative to the legs and the hip. Therefore, when the urine receiving pad 100 is attached to the male genitalia P, the urine receiving pad 100 is fixed in an inclined position, as shown in FIG. 10.

If, for example, a wearer wears a diaper 104 (or underwear) in this condition, as shown in FIG. 10, the bottom portion 103 of the urine receiving pad 100 is brought into contact with the diaper 104 and thus partly folded, which may cause a large wrinkle in the urine receiving pad 100 to close an internal space of the urine receiving pad 100. In this case, urine excreted from the male genitalia may overflow from the opening 101.

The present invention is made in view of the circumstances described above, and an object of the present invention is to provide a technique of suppressing an overflow of urine from an opening of a urine receiving pad.

### Solution to Problem

To solve the above-described problem, according to a first aspect, there is provided a urine absorbing article attached to a male genitalia to absorb urine excreted from the male genitalia. The urine absorbing article includes a bag body which has a liquid-permeable top sheet forming an inside surface, a liquid-impermeable back sheet forming an outside surface, and an absorber interposed between the top sheet and the back sheet. The bag body has an opening through which the male genitalia is inserted into the bag body. The bag body includes a side edge portion extending in a first direction, and a top edge portion extending in a second direction which intersects with the first direction, and connected to the side edge portion. The opening is provided in the side edge portion at a position deviated toward the top edge portion side.

According to a second aspect, in the urine absorbing article according to the first aspect, the first direction is a lengthwise direction of the bag body.

According to a third aspect, in the urine absorbing article according to the first or second aspect, the opening is provided so as to extend over both of the side edge portion and the top edge portion, and an opening adjustment part is provided at the top edge portion, the opening adjustment part adjusting an opening size of the opening by putting opposed portions of the bag body which form the opening one over the other and fixing them in this state.

According to a fourth aspect, in the urine absorbing article according to any one of the first to third aspects, at least a part of the side edge portion where the opening is provided has an overhang portion which protrudes outwardly along the second direction.

According to a fifth aspect, in the urine absorbing article according to any one of the first to fourth aspects, a bottom portion of the bag body has a folded portion folded into the bag body.

According to a sixth aspect, in the urine absorbing article according to the fifth aspect, the absorber is arranged in the folded portion.

According to a seventh aspect, in the urine absorbing article according to any one of the first to sixth aspects, the bag body further includes a biasing portion which biases the bag body so as to contract the bag body.

According to an eighth aspect, in the urine absorbing article according to the seventh aspect, the biasing portion includes an elastic member which is attached around the bag body while being expanded.

According to a ninth aspect, in the urine absorbing article according to any one of the first to eighth aspects, a portion near the opening has a color different from that of the circumference thereof.

### Advantageous Effects of Invention

In the urine absorbing article according to the first to ninth aspects, since the male genitalia can be inserted through the opening formed in the side edge portion, a portion near the bottom portion of the bag body can be prevented from being largely folded when a diaper, underwear, or the like, is worn. Thus, occurrence of a relatively large wrinkle in the bag body is suppressed. This can reduce the possibility that an internal space of the bag body is closed, so that an overflow of urine from the opening can be suppressed.

Particularly, in the urine absorbing article according to the second aspect, a large space for accommodating urine can be ensured below the opening within the bag body.

Particularly, in the urine absorbing article according to the third aspect, an opening size of the opening is adjusted by the opening adjustment part, to thereby make it difficult that the male genitalia is detached from the bag body. Moreover, since the bag body can be firmly attached to the male genitalia, occurrence of urine leakage can be reduced.

Particularly, in the urine absorbing article according to the fourth aspect, when the urine absorbing article is attached, the male genitalia is enveloped by the overhang portion, to make it difficult that the male genitalia is detached from the bag body. Moreover, since a characteristic structure of partially protruding is formed, the position of an insertion opening to which the male genitalia is to be inserted is easily recognizable.

Particularly, in the urine absorbing article according to the fifth aspect, a urine storage space can be expanded while increase of the size of the bag body is suppressed. Thus, the urine capacity relative to the size of the bag body is improved.

Particularly, in the urine absorbing article according to the sixth aspect, since a place where the absorber is arranged is enlarged, the urine absorbing capability can be improved. When urine is excreted, the absorber of the folded portion absorbs the excreted urine, so that the internal space of the bag body is enlarged. This can expand the urine storage space.

Particularly, in the urine absorbing article according to the seventh aspect, the bag body is pulled inwardly, to make it easy that the bag body swells into a pouched shape. This can prevent the internal space of the bag body from being closed. Thus, a urine absorption efficiency of the urine absorbing article can be improved.

Particularly, in the urine absorbing article according to the eighth aspect, the bag body can be easily contracted.

In the urine absorbing article according to the ninth aspect, the position of the opening of the bag body can be easily recognized based on the color.

These and other objects, features, aspects and advantages of the present invention will become more apparent from the following detailed description of the present invention when taken in conjunction with the accompanying drawings.

### BRIEF DESCRIPTION OF DRAWINGS

FIG. 1 is an overall view of a urine receiving pad according to a preferred embodiment.

FIG. 2 is a cross-sectional view of the urine receiving pad as taken along the line II-II of FIG. 1.

FIG. 3 is a cross-sectional view of a bag body as taken along the line III-III of FIG. 2.

FIG. 4 is a developed view of the urine receiving pad.

FIG. 5 shows a state where the urine receiving pad is attached to a male genitalia.

FIG. 6 shows the urine receiving pad attached to the male genitalia while a diaper is additionally attached.

FIG. 7 is a cross-sectional view of a urine receiving pad according to a modified example.

FIG. 8 is a cross-sectional view of a urine receiving pad according to another modified example.

FIG. 9 is an overall view of a urine receiving pad according to still another modified example.

FIG. 10 is an overall view of a conventional urine receiving pad.

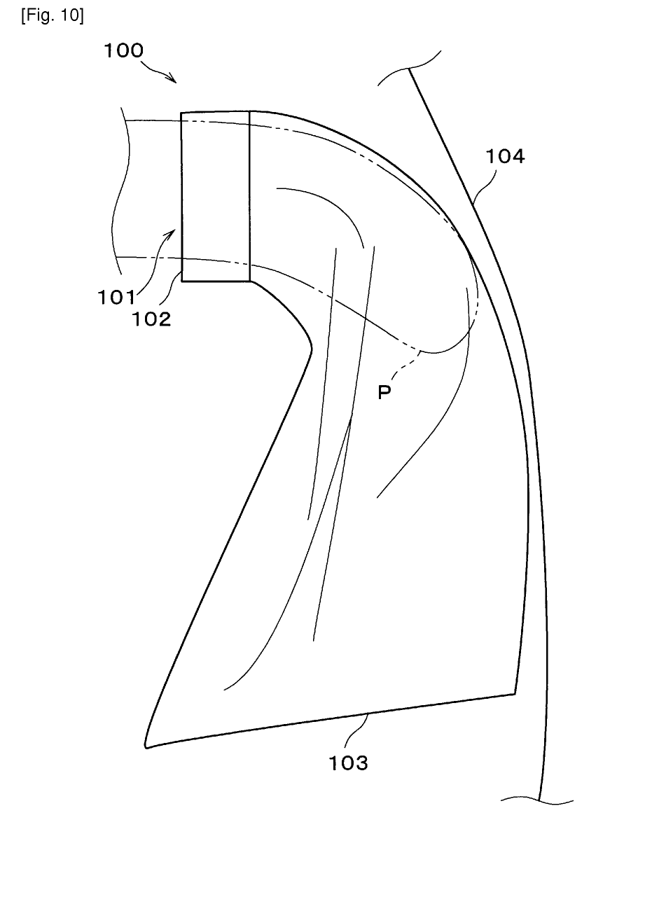

### DESCRIPTION OF EMBODIMENTS

Hereinafter, a preferred embodiment will be described with reference to the accompanying drawings.

### 1. PREFERRED EMBODIMENT

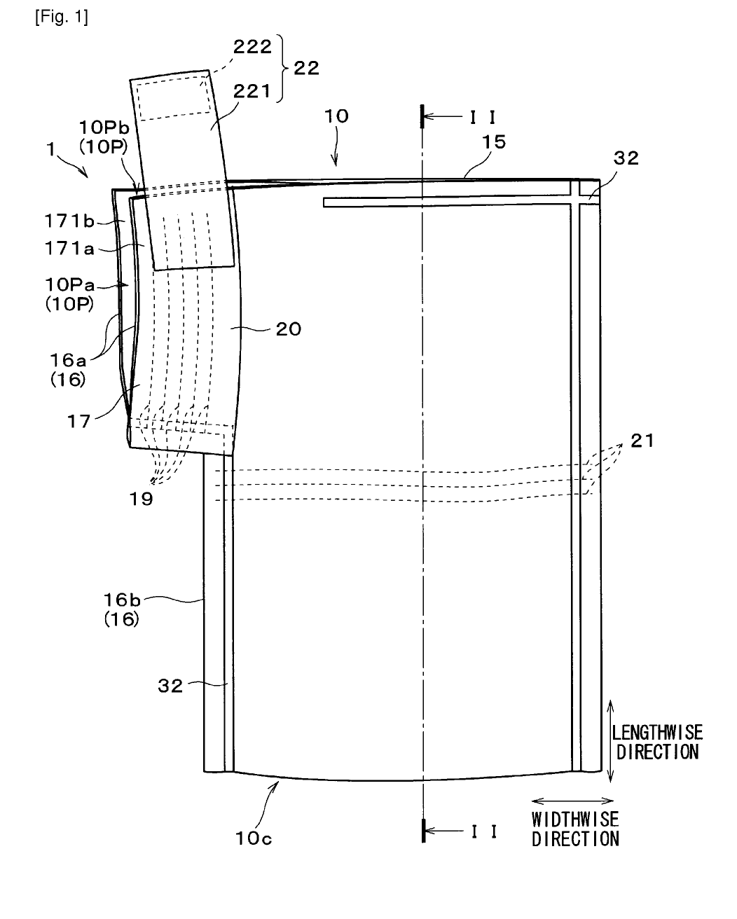

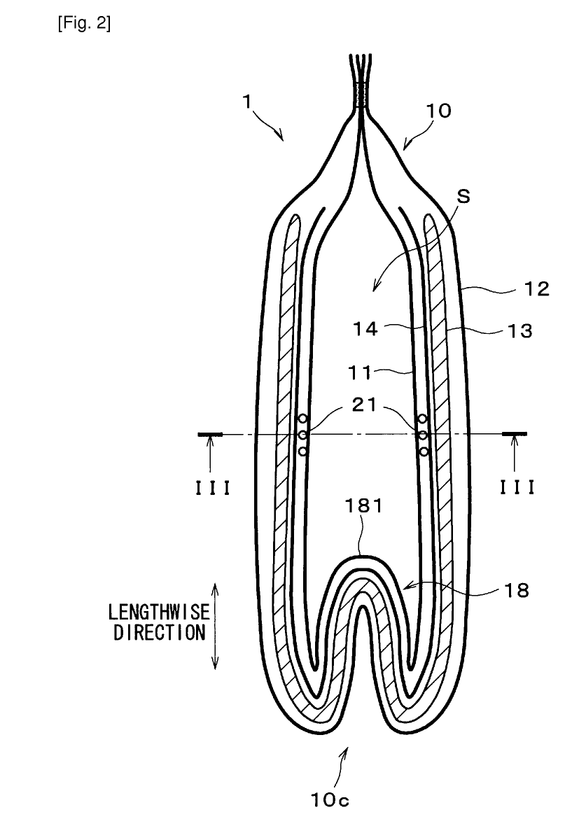

FIG. 1 is an overall view of a urine receiving pad 1 according to a preferred embodiment. FIG. 2 is a cross-sectional view of the urine receiving pad 1 as taken along the line II-II of FIG. 1. The urine receiving pad 1 is a urine absorbing article which is attached to a male genitalia (penis) and absorbs urine excreted from the male genitalia. A wearer wears the urine receiving pad 1, and can additionally wear an outer equipment such as a diaper and underwear over the urine receiving pad 1. When the urine receiving pad 1 is worn, in a case of a small amount of urination, replacement of only the urine receiving pad 1, without replacement of diaper or the like, suffices. This can reduce the economic burden on a wearer and the care burden on a caregiver, and moreover is environment-friendly.

As shown in FIG. 1, the urine receiving pad 1 of this preferred embodiment has a bag body 10 formed by substantially rectangular surfaces being laminated on each other. As shown in FIG. 2, the bag body 10 has a top sheet 11 forming an inside surface thereof, a liquid-impermeable back sheet 12 forming an outside surface thereof, and an absorber 13 interposed between the top sheet 11 and the back sheet 12.

Urine excreted within the bag body 10 can permeate the top sheet 11 under ordinary pressure, but cannot permeate the back sheet 12. The urine is absorbed and held by the absorber 13 between the top sheet 11 and the back sheet 12.

The top sheet 11 is made of a nonwoven fabric using hydrophilic fibers (cellulose, rayon, cotton, or the like), for example. Alternatively, the top sheet 11 may be made of a nonwoven fabric using hydrophobic fibers (polypropylene, polyethylene, polyester, polyamide, nylon, or the like) whose surfaces are treated with a surfactant to make them liquid-permeable.

The back sheet 12 is made of a water-repellent nonwoven fabric using hydrophobic fibers (polypropylene, polyethylene, polyester, polyamide, nylon, or the like), for example. The back sheet 12 may be made of a plastic film, or alternatively may be made of a plastic film and a highly flexible nonwoven fabric attached to the outside thereof.

The absorber 13 is made of a granular polymer absorber, for example. As shown in FIG. 2, in this preferred embodiment, the polymer absorber is fixed to an inside surface of the back sheet 12 with an adhesive (not shown), and a liquid-permeable auxiliary sheet 14 for covering the polymer absorber is provided at the inner side of the polymer absorber. Thus, the polymer absorber is distributed in a sheet-like manner.

Here, the absorber 13 may be held between the top sheet 11 and the back sheet 12 without interposition of the auxiliary sheet 14. The absorber 13 may be formed by a mass obtained by mixing a granular polymer absorber with a layer of aggregation of pulverized hydrophilic fibers such as pulp fibers or cellulose fibers covered with a paper sheet such as a tissue paper or a liquid-permeable nonwoven fabric sheet. However, in order to suppress bulkiness of the absorber 13, it is desirable that the absorber 13 is formed of a polymer absorber without a pulp.

As shown in FIG. 1, the bag body 10 has a top edge portion 15 extending in a widthwise direction, and a side edge portion 16 extending in a lengthwise direction and forming one side edge of the bag body 10. On the side edge portion 16, an overhang portion 17 protruding in the widthwise direction is formed at a position of an upper portion of the bag body 10 (a portion of the bag body 10 at the side opposite to a bottom portion 10c). The side edge portion 16 of the bag body 10 is constituted by a side edge portion 16a which corresponds to a side edge of the overhang portion 17, and a side edge portion 16b which corresponds to a part of the side edge portion 16 except the overhang portion 17.

Although in this preferred embodiment, the length of the top edge portion 15 is smaller than the length of the side edge portion 16, the length of the top edge portion 15 may be larger than the length of the side edge portion 16. However, by increasing the length of the side edge portion 16, a large space for accommodating urine can be ensured at the lower side of an opening 10P within the bag body 10, and a larger amount of the absorber 13 can be arranged.

A slit extending in the lengthwise direction is formed in the side edge portion 16a of the overhang portion 17, so that a first opening 10Pa opening in the widthwise direction is formed. Additionally, a slit extending in the widthwise direction is formed in an upper portion of the overhang portion 17, so that a second opening 10Pb opening in the lengthwise direction is formed. The slit in the top edge portion 15 and the slit in the side edge portion 16 are connected to each other, and the first opening 10Pa and the second opening 10Pb form a single opening 10P. The slits forming the opening 10P divide the overhang portion 17 into right and left parts, namely, a side surface portion 171a and a side surface portion 171b.

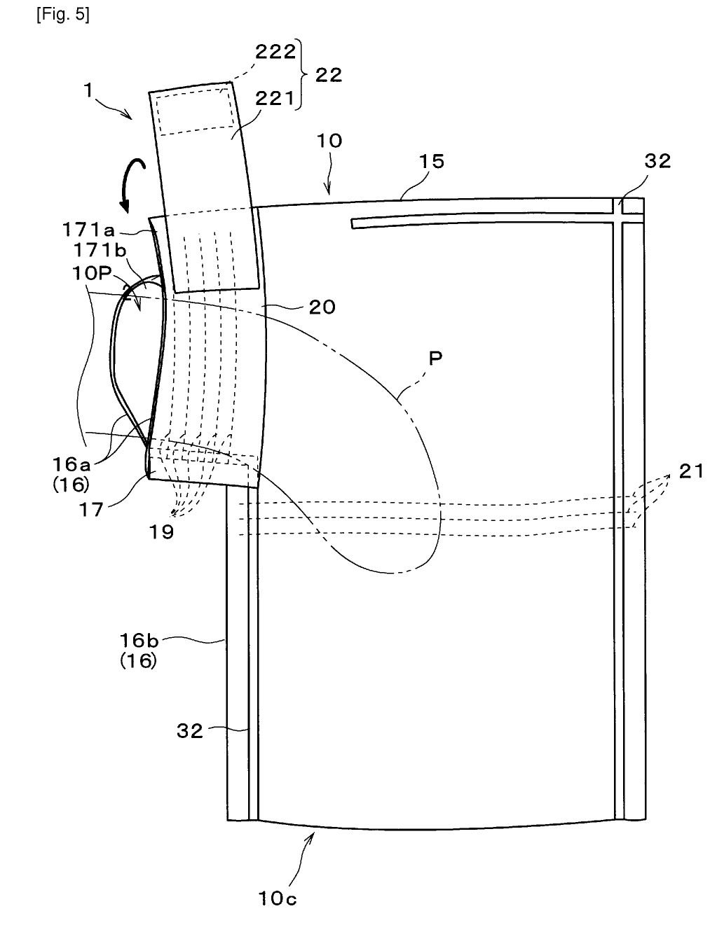

Due to the presence of the opening 10P, the inside space of the bag body 10 communicates with the outside thereof. A male genitalia P is inserted into the bag body 10 through, in the opening 10P, the opening 10Pa in the side edge portion 16 (see FIG. 5). In the bag body 10, since the second opening 10Pb is formed, the opening 10P can be opened widely, to make the insertion of the male genitalia P easy. Moreover, since the bag body 10 has the overhang portion 17 with a characteristic structure, the wearer or the caregiver can easily recognize where the male genitalia P is to be inserted.

As shown in FIG. 2, the bottom portion 10c (at the side opposite to the top edge portion 15) of the bag body 10 is folded into the bag body 10, to form a folded portion 18. The folded portion 18 is formed by the top sheet 11, the auxiliary sheet 14, the absorber 13, and the back sheet 12 being folded into the bag body 10.

Since the folded portion 18 is formed in the urine receiving pad 1, the inside surface of the bag body 10 obtains a large surface area while increase of the size of the bag body 10 with respect to the lengthwise direction is suppressed. Additionally, since such a folded portion 18 is formed, an increased amount of the absorber 13 can be held between the top sheet 11 and the back sheet 12.

Even before urination, the bottom portion 10c of the bag body 10 is kept slightly expanded because of the bulkiness and a slight elasticity of the respective parts of sheets included in the folded portion 18. Therefore, also in the upper portion of the bag body 10, a contact between parts of the inside surface does not easily occur, so that a larger space can be ensured within the bag body 10.

When urine is excreted into the bag body 10, the absorber 13 absorbs the urine and swelled. This unfolds the folded portion 18, and thus the bottom portion 10c of the bag body 10 is further expanded. Consequently, a still larger space is ensured within the bag body 10.

Since the urine receiving pad 1 has the folded portion 18, the bag body 10 can obtain a large internal space. This can reduce the possibility that when the wearer excretes urine, unabsorbed part of the urine flows out from the bag body 10.

In this preferred embodiment, the folded length of the folded portion 18 is set equal to or less than half the size of the bag body 10 with respect to the lengthwise direction. A space S capable of accommodating the whole of the male genitalia is ensured within the bag body 10 at the top edge portion 15 side relative to a top 181 of the folded portion 18. This makes it easy that the urine excreted from the male genitalia P of the wearer is directly in contact with the top 181 of the folded portion 18. Thus, the folded portion of the absorber 13 can be efficiently swelled, and therefore the bottom portion 10c of the bag body 10 can be expanded in a good manner.

Referring to FIG. 1 again, the overhang portion 17 has a plurality of string-like elastic members 19 extending along the lengthwise direction. At the overhang portion 17, an end portion of the back sheet 12 which forms the outer surface of the bag body 10 is folded into an inner side of an edge of the opening 10P. The elastic members 19 are provided within this folded portion of the back sheet 12. In other words, the elastic members 19 are buried with both sides thereof being sandwiched by the back sheet 12.

The plurality of elastic members 19 are, while being expanded beyond their natural length, attached to the back sheet 12 with an adhesive or the like. Therefore, the plurality of elastic members 19 cause an elastic force that shrinks the opening 10P (that is, reduce the size of the opening). On the other hand, the wearer of the urine receiving pad 1 or the caregiver can easily expand the opening 10P in the lengthwise direction against the elastic force of the elastic member 19. The elastic member 19 can be made of a polyurethane yarn for example, but other materials such as a polyurethane film and a natural rubber may be used.

Furthermore, an opening sheet 20 is attached to the opening 10P of the overhang portion 17. The opening sheet 20 is attached with an adhesive or the like so as to extend from the inside surface of the top sheet 11 to the outside surface of the back sheet 12. The opening sheet 20 serves to soften a texture to the skin of the wearer to reduce skin irritation. The opening sheet 20 is, for example, made of a nonwoven fabric using relatively soft fibers, but not limited to the nonwoven fabric.

As shown in FIGS. 1 and 2, a plurality of string-like elastic members 21 extending in the widthwise direction are arranged near a central portion of the bag body 10 with respect to the lengthwise direction. The plurality of elastic members 21 are positioned between the top sheet 11 and the auxiliary sheet 14. The plurality of elastic members 21 are, while being expanded beyond their natural length, attached to the top sheet 11 (or the auxiliary sheet 14) via an adhesive. The plurality of elastic members 21 are provided in each of both side surfaces of the bag body 10, so as to extend over at least the absorber 13 in the widthwise direction.

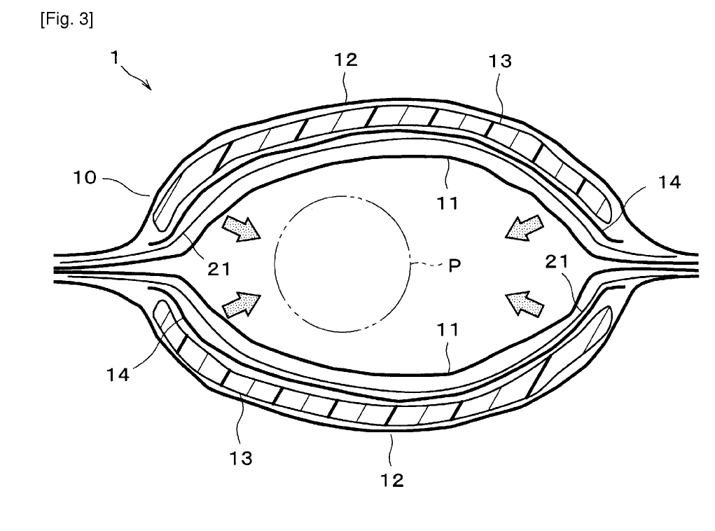

FIG. 3 is a cross-sectional view of the bag body 10 as taken along the like III-III of FIG. 2. As shown in FIG. 3, a portion where the elastic members 21 are arranged and a neighborhood thereof are contracted in the widthwise direction due to the elastic force of the elastic members 21. That is, the elastic members 21 bias the bag body 10 so as to contract the bag body 10 in the widthwise direction. Since the elastic members 21 are provided substantially over the entire width of the bag body 10, both end portions of the bag body 10 are pulled toward the inside of the bag body 10, as indicated by the arrows of FIG. 3. Therefore, the bag body 10 easily swells into a pouched shape such that both side surfaces of the bag body 10 protrude outwardly.

Although in this preferred embodiment, the elastic members 21 are provided so as to extend in the widthwise direction, the elastic members 21 may be arranged so as to extend in any direction. When the elastic members 21 are provided so as to extend in any direction, the bag body 10 is contracted in this direction, and thus the bag body 10 easily swells into a pouched shape.

It is preferable that the elastic members 21 are provided at a portion where the absorber 13 is arranged. Since the portion where the absorber 13 is arranged has higher rigidity than that of the other portions, loss of shape of the bag body 10 due to the elastic members 21 can be suppressed.

The elastic members 21 may be provided between the back sheet 12 and the absorber 13. However, to avoid weakening adhesive force between the absorber 13 and the auxiliary sheet 14, the position at which the elastic members 21 are provided between the top sheet 11 and the auxiliary sheet 14 than between the back sheet 12 and the absorber 13.

A fixing member 22 (opening adjustment part) is provided near an edge of the opening 10P. The fixing member 22 fixes the size of the opening 10P with respect to the lengthwise direction, in accordance with the size of the male genitalia P. The fixing member 22 has a base sheet 221 and a male member 222. The base sheet 221 extends in the lengthwise direction of the bag body 10, and has its base end portion attached to the side surface portion 171a of the overhang portion 17 with an adhesive or the like. The male member 222 is provided near the other end portion of the base sheet 221.

The male member 222 is constituted by, for example, a hook-and-loop fastener including a hook which serves as a fastening member. The male member 222 is attachable to and detachable from any position on the surface of the opening sheet 20. In a case where the back sheet 12 is made of a nonwoven fabric, the male member 222 is also attachable to and detachable from any position of the back sheet 12.

The fixing member 22 may not necessarily be configured such that the male member 222 is attached to the opening sheet 20. For example, a female member (for example, a loop of a hook-and-loop fastener) may be provided at a portion on the outside surface of the back sheet 12 near the opening 10P, and the male member 222 may be adhered to the female member. An adhesive tape may be adopted as the male member 222.

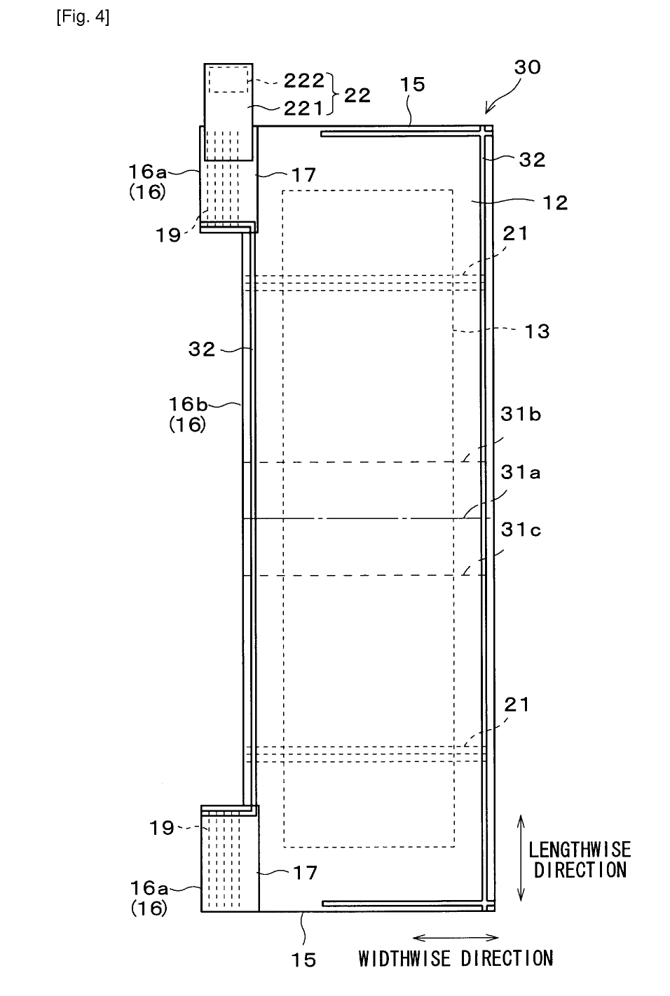

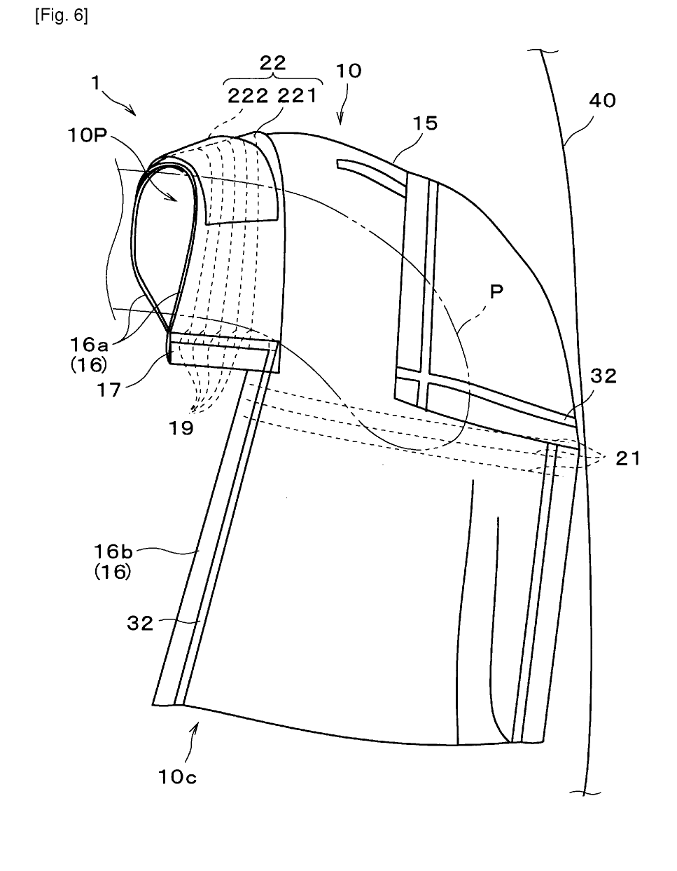

FIG. 4 is a developed view of the urine receiving pad 1. The urine receiving pad 1 is formed as a substantially rectangular laminated body 30 in which the top sheet 11, the auxiliary sheet 14, the absorber 13, and the back sheet 12 are laminated in the mentioned order. Here, the laminated body 30 is formed such that a portion serving as the overhang portion 17 (upper and lower edge portions at the left side edge in FIG. 6) protrudes in the widthwise direction.

To manufacture the urine receiving pad 1, firstly, the laminated body 30 is folded so as to bring the top sheet 11 to the inside and so as to form the folded portion 18. More specifically, as shown in FIG. 4, while the back sheet 12 is facing upward, the laminated body 30 is valley-folded so as to protrude backward, at a fold line 31a which extends in the widthwise direction at a central portion of the laminated body 30 with respect to the lengthwise direction. Additionally, the laminated body 30 is mountain-folded so as to protrude upward, at fold lines 31b and 31c which extend in the widthwise direction at opposite sides of the fold line 31a.

In the laminated body 30 folded in this manner, a seal region 32 is bonded by a heat seal or the like. At this time, a seal-bonding is not performed at a portion corresponding to the side edge portion 16a of the overhang portion 17 and at a portion corresponding to a part of the top edge portion 15 at the overhang portion 17 side. Thus, the opening 10P is formed therein.

In this preferred embodiment, a part of the laminated body 30 serving as the folded portion 18 is folded, and then the bonding by the heat seal is performed. This makes it easy to form the folded portion 18. Portions at both sides of the folded portion 18 are also fixed by the heat seal while being folded inwardly. This can prevent the folded portion 18 folded inwardly from spontaneously unfolding toward the outside of the bag body 10.

FIG. 5 shows a state where the urine receiving pad 1 is attached to the male genitalia P. To attach the urine receiving pad 1, firstly, the wearer or the caregiver expands the opening 10P of the urine receiving pad 1. Then, the male genitalia P is inserted through the first opening 10Pa of the opening 10P which is at the side edge portion 16a side.

In this state, the wearer or the caregiver overlays the right and left parts of the overhang portion 17 divided by the opening 10P, namely, the side surface portions 171a and 171b, one over the other, and thereby stores the male genitalia P within the bag body 10. At this time, the side surface portion 171b not having the fixing member 22 is put between the male genitalia P and the side surface portion 171a having the fixing member 22. By making a lower part of the male genitalia P in contact with the lower end of the overhang portion 17, occurrence of a gap between the opening 10P and the male genitalia P can be prevented.

The fixing member 22 is, while being pulled, wound around the opening 10P through which the male genitalia P is inserted, and additionally the male member 222 of the fixing member 22 is attached to the side surface portion 171b. In this manner, the size of the opening 10P is adjusted in accordance with the size of the male genitalia P, so that the urine receiving pad 1 is firmly fixed to the male genitalia P. This makes it difficult that the excreted urine is leaked from the opening 10P.

In this preferred embodiment, since the opening 10P is provided at the overhang portion 17, the base sheet 221 of the fixing member 22 can be wound around the overhang portion 17 in accordance with the size of the male genitalia P, so that the urine receiving pad 1 can be firmly fixed to the male genitalia P. However, the overhang portion 17 is not essential, and may be omitted.

FIG. 6 shows the urine receiving pad 1 attached to the male genitalia P while a diaper 40 is additionally attached. As shown in FIG. 6, in the urine receiving pad 1 of this preferred embodiment, an insertion direction of the male genitalia P is the widthwise direction of the bag body 10. Accordingly, when the urine receiving pad 1 is attached, the bottom portion 10c of the bag body 10 tends to be positioned below the male genitalia P. This can prevent the bottom portion 10c and a portion therearound from being folded when the diaper 40 or the like is additionally attached. Therefore, a passage extending to the bottom portion 10c can be easily ensured, which can effectively prevent the urine excreted from the male genitalia P from leaking from the opening 10P.

The length of the bag body 10 with respect to the widthwise direction is smaller than the length thereof with respect to the lengthwise direction. Thus, the size of a wrinkle occurring in the bag body 10 is smaller when the bag body 10 is folded in the widthwise direction than when the bag body 10 is folded in the lengthwise direction. Therefore, the excreted urine can be prevented from leaking from the opening 10P.

In the urine receiving pad 1 of this preferred embodiment, the opening 10P is provided at the side edge portion 16a. Thereby, when the bag body 10 is pulled downward because of influence of the weight of the urine, an upper portion of the inside of the bag body 10 tends to be hooked on the male genitalia P. Therefore, as compared with the conventional urine receiving pad 100 in which the opening is provided at the top edge portion, the bag body 10 is hardly detached from the male genitalia P.

### 2. MODIFIED EXAMPLE

Although the preferred embodiment has been described above, the present invention is not limited to the above-described preferred embodiment. In the following, components having the same functions as in the preferred embodiment described above are denoted by the common reference numerals, and descriptions thereof are omitted, as appropriate.

For example, although in the above-described preferred embodiment, the absorber 13 is interposed substantially throughout the space between the top sheet 11 and the back sheet 12, there may be a region where the absorber 13 is not interposed between the top sheet 11 and the back sheet 12. However, in order to obtain the effect of expanding the bottom portion of the bag body 10 at the time of urination, it is desirable that the absorber 13 is arranged at least at the folded portion 18.

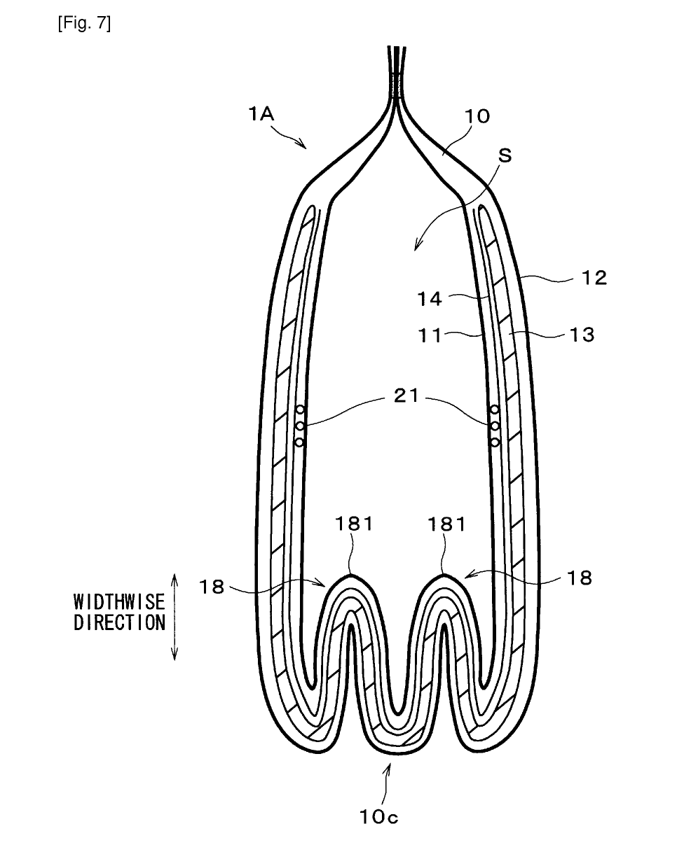

In the above-described preferred embodiment, one folded portion 18 is provided at the bottom portion of the bag body 10. However, a plurality of folded portions may be provided at the bottom portion of the bag body 10. For example, as in a urine receiving pad 1A shown in FIG. 7, two folded portions 18 may be provided at the bottom portion of the bag body 10. This configuration can further increase the surface area of the inside surface of the bag body 10, and moreover increase the amount of the absorber 13 interposed between the top sheet 11 and the back sheet 12. Furthermore, a larger space can be ensured within the bag body 10.

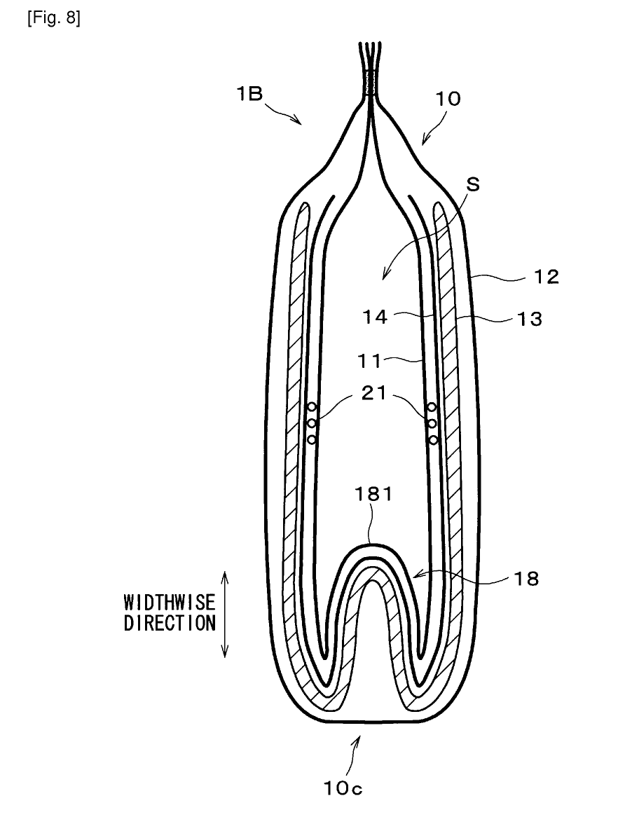

In the above-described preferred embodiment, the top sheet 11, the auxiliary sheet 14, the absorber 13, and the back sheet 12 are folded into the bag body 10 at the folded portion 18. However, it is not always necessary that all of these sheets are folded. For example, as in a urine receiving pad 1B shown in FIG. 8, only the top sheet 11, the auxiliary sheet 14, and the absorber 13 may be folded into the bag body 10. In this configuration as well, the surface area of the inside surface of the bag body 10 and the amount of the absorber 13 can be increased, and moreover the bottom portion of the bag body 10 can be kept slightly expanded, so that a space can be ensured within the bag body 10. However, in order to expand the bottom portion of the bag body 10 at the time of urination, it is desirable that all the sheets including the back sheet 12 are folded into the bag body 10.

In the above-described preferred embodiment, the overhang portion 17 is provided at a part of the side edge portion 16 of the bag body 10. However, this overhang portion 17 may not necessarily be provided. For example, the side edge portion 16 may have a linear shape, and the first opening 10Pa may be formed at the side edge portion 16 having the linear shape.

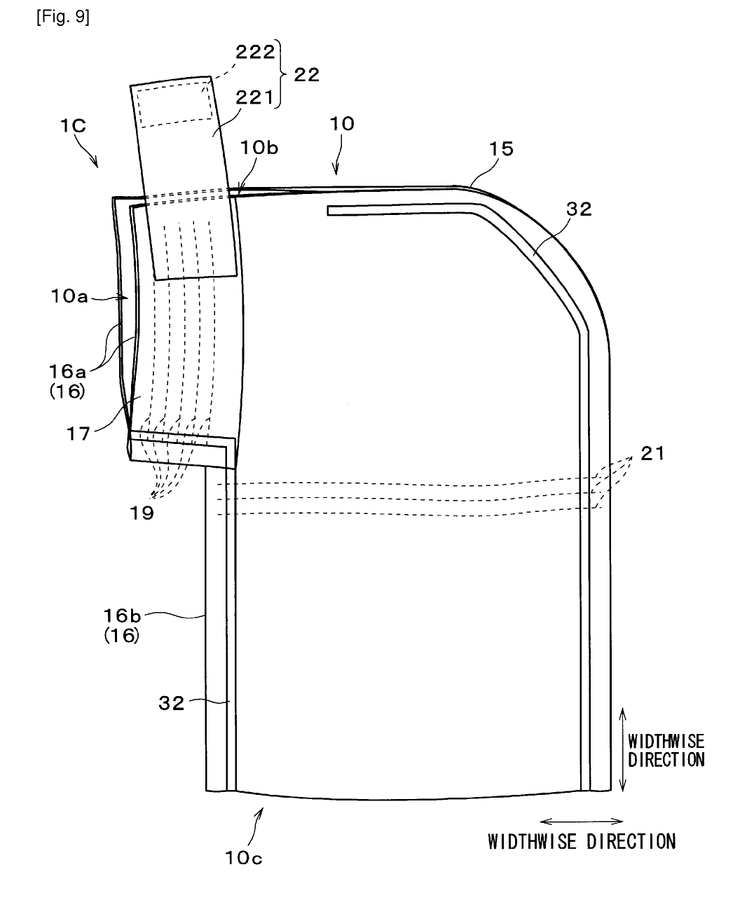

As shown in FIG. 6, in the bag body 10 of the above-described preferred embodiment, a corner portion at which the top edge portion 15 intersects with a side edge portion at the side opposite to the opening 10P side may be folded by being in contact with a diaper or the like. Therefore, as in a urine receiving pad 1C shown in FIG. 9, the corner portion which may be in contact with the diaper 40 may be cut off in advance. This can suppress folding of the bag body 10, and thus suppress the bulkiness of the urine receiving pad 1 within the diaper 40 or the like. The corner portion may be cut off into a curved shape or a linear shape, and may have any shape.

In the above-described preferred embodiment, the laminated body 30 is folded to form the folded portion 18, and then the bonding by the heat seal is performed. However, it may be acceptable that the laminated body 30 is half-folded and both side edges 16 and the top edge portion 15 are sealed, and then the bottom portion 10c of the bag body 10 is folded into the bag body 10 to form the folded portion 18.

In the above-described preferred embodiment, since the overhang portion 17 is provided, the wearer or the caregiver can easily recognize the position of the opening 10P through which the male genitalia P is to be inserted. Here, to make the position of the opening 10P recognizable, marker means allowing the position of the opening to be visually recognized may be provided to the bag body. For example, a portion near the opening 10P may be painted with a color different from that of the circumference thereof, or the opening may be indicated by an arrow or the like.

In the above-described preferred embodiment, the rectangular laminated body 30 is folded and both side edges thereof are sealed, thereby forming the bag body 10. However, the bag body 10 may not necessarily be formed as a single laminated body 30. For example, the bag body 10 may be formed by bonding a plurality of laminated bodies to each other.

While the invention has been described in detail, the foregoing description is in all aspects illustrative and not restrictive. It is therefore understood that numerous modifications and variations can be devised without departing from the scope of the invention.

1. A urine absorbing article attached to a male genitalia to absorb urine excreted from the male genitalia,
said urine absorbing article comprising a bag body which has a liquid-permeable top sheet forming an inside surface, a liquid-impermeable back sheet forming an outside surface, and an absorber interposed between said top sheet and said back sheet, said bag body having an opening through which said male genitalia is inserted into said bag body,
said bag body including:
a side edge portion extending in a first direction; and
a top edge portion extending in a second direction which intersects with said first direction, and connected to said side edge portion,
said opening being provided in said side edge portion at a position deviated toward a said top edge portion side.

2. The urine absorbing article according to claim 1, wherein
said first direction is a lengthwise direction of said bag body.

3. The urine absorbing article according to claim 1, wherein
said opening is provided so as to extend over both of said side edge portion and said top edge portion,
an opening adjustment part is provided at said top edge portion, said opening adjustment part adjusting an opening size of said opening by overlaying opposed portions of said bag body which form said opening and fixing them in this state.

4. The urine absorbing article according to claim 1, wherein
at least a part of said side edge portion where said opening is provided has an overhang portion which protrudes outwardly along said second direction.

5. The urine absorbing article according to claim 1, wherein
a bottom portion of said bag body has a folded portion folded into said bag body.

6. The urine absorbing article according to claim 5, wherein
said absorber is arranged in said folded portion.

7. The urine absorbing article according to claim 1, wherein
said bag body further includes a biasing portion which biases said bag body so as to contract said bag body.

8. The urine absorbing article according to claim 7, wherein
said biasing portion includes an elastic member which is attached around said bag body while being expanded.

9. The urine absorbing article according to claim 1, wherein
a portion near said opening has a color different from that of the circumference thereof.
